(19) United States
(12) Patent Application Publication
(10) Pub. No.: US 2003/0120282 A1
(43) Pub. Date: Jun. 26, 2003

(54) Stereotaxic manipulator with retrofitted linear scales and digital display device

(76) Inventors: Charles W. Scouten, Downers Grove, IL (US); James G. Unnerstall, O'Fallon, MO (US); Brian J. Forinash, Louis, MO (US)

Correspondence Address: Patrick D. Kelly, 11939 Manchester # 403, St. Louis, MD, 63131, US

(21) Appl. No.: 10/036,231

(22) Filed: Dec. 24, 2001

(51) Int. Cl. A61B 19/00

(52) U.S. Cl. 606/130

(57) ABSTRACT

A convenient low-cost system is disclosed for digitally displaying instrument locations during a stereotaxic procedure on an animal such as a rat or mouse. This system, which can be retrofitted onto most types of existing stereotaxic manipulators, uses three optical or capacitance scaling devices, mounted orthogonally. An electronic reader head interacts with each scaling device, to measure the position and travel of the manipulator along the medial-lateral (X), anterior-posterior (Y), and dorsal-ventral (Z) axes. Each reader head sends electronic signals to a small display box that can be placed or mounted in any convenient location, or to a computer with a monitor, and all three coordinates are displayed continuously. This system provides a simple zeroing function, which can set all values to zero when an instrument tip reaches a fixed location such as the bregma point on a skull. “Data-grab” capabilities allow the system to record and/or print the coordinates of the instrument at any step or time; if a computer is used, complex data-handling can be provided. An inexpensive system provides a resolution of 5 microns, which is roughly half the diameter of a typical cell; finer resolutions (such as 1 micron) can also be provided. This system is not bulky or cumbersome, and allows the use of optical microscopes and/or video cameras with magnifying lenses for video recording and real-time video displays on a nearby monitor.

### BACKGROUND OF THE INVENTION

This invention relates to equipment used in biological and medical research, and in particular to neurological research which uses small animals, such as rats or mice.

Numerous types of biological and medical research require that the head and/or spine of a rat, mouse, or other small mammal must be held in a secure and stationary position throughout the duration of a surgical or similar procedure. One major category of such research, which is discussed throughout this text as an example but which is not intended to be limiting, involves invasive neurological procedures carried out on rats or mice, which are widely used in neurology research because of low cost, ease of breeding and care, and recent advances in genetic analysis and manipulation. Such tests may involve, for example, implantation of a stimulatory or measuring electrode in a specific targeted region of the brain, or insertion of a microscopic needle to inject a test compound into a targeted brain region. When these types of experiments are done, the head of the rat or other small animal (for convenience, all references below refer only to rats) must be held in a totally immobilized position.

To accomplish that type of immobilization, neurology researchers typically use devices called “stereotaxic holders” during an invasive procedure. These types of holders are commercially available from several companies, such as myNeurolab, Inc. (St. Louis, Mo.), David Kopf Instruments (Tujunga, Calif.), and Stoelting Company (Wood Dale, Ill.).

A typical stereotaxic holder as used in the prior art, designed for holding a rat or other small animal, is shown in FIG. 1. The main components of this type of stereotaxic holder assembly 100 include a base plate 102, which is large enough to completely support a rat's entire body on top of it, and heavy enough to prevent or minimize any motion if a worker inadvertently bumps or jostles it during a test; and a “U-frame” 104, which is supported at an appropriate height by posts 105-107.

During use, the animal's head is held securely in place by a combination of two “ear bars” (also called ear pins) and a snout clamp 121. Ear bars 110 and 112 can be adjusted by sliding them, in either direction, through loosened clamps 111 and 113, mounted at the two ends of the U-frame 104. After an animal has been secured in the holder and the ear bars 110 and 112 have been properly and securely positioned, they are held immobile by tightening clamp screws 114 and 116. The snout clamp assembly allows the animal's nose and upper jaw to be placed on top of a horizontal plate 120, with its upper front teeth projecting downward into an orifice in plate 120. The snout (and the entire head, as a result) is then immobilized by lowering horizontal clamping bar 121 down until it firmly presses against on top of the animal's snout. Various adjusting components, including slidable plate 130, which can be held in a desired position by knob 131, and vertical slide assembly 132, allow the snout clamp to be placed at a range of positions and angles, depending on the type of animal being secured, and the type of procedure being done.

In most stereotaxic holders used in research laboratories, the components listed above merely hold an animal's head in a fixed position, while an invasive procedure is done to the head or brain, spinal cord, or other anatomical structure. In most cases, the procedure uses one or more devices which directly contact the animal's head or brain, such as one or more needles, blades, electrode tips, patch clamps, etc. For convenience, such devices are referred to herein as “instruments”.

Similarly, for convenience, any manipulation or intervention involving an animal being held in a stereotaxic holder is referred to herein as a procedure, invasive procedure, test, or similar terms. Although the word “test” is used herein for convenience, it should be recognized that in many situations, the analytical test(s) which will evaluate the effects of a surgical or other invasive procedure may not be carried out until days, weeks, or even months later.

The degree of control needed for invasive procedures on a rat or mouse are usually measured in either: (i) tenths of a millimeter, for procedures carried out with the naked eye; or, (ii) microns, for procedures carried out with the aid of a microscope. For reference, a micron is 1/1,000 of a millimeter. Most mammalian cells have diameters that are in the general range of about 10 microns, which is 1/100 of a millimeter.

Since unaided hands cannot provide the degree of precision control needed for most types of invasive neurological procedures, a “manipulator” assembly (shown as assembly 200 in FIG. 1, and in greater detail in FIG. 2) is used to control and move instruments in a careful and precise manner.

A typical manipulator 200 as used in a stereotaxic holder 100 is mounted on a sliding base 180, which is held within a nonmoving base component 184, which is screwed or bolted tightly to one arm of the U-frame 104. Fine control of the movement and positioning of the sliding base 180 is provided by rotating knob 182, which is coupled to a threaded shaft (not shown) that is positioned inside, beneath, or adjacent to slide 180. The threaded shaft rotates within a non-movable bushing that is securely affixed to the nonmoving base component 184; accordingly, when the threaded shaft is rotated by a human operator, by using knob 182, the slide 180 and the entire movable manipulator assembly 200 can move in either an anterior direction (i.e., in the direction from the animal's tail, toward its nose) or a posterior direction (i.e., from the animal's nose, toward its tail).

As used herein, the base and slide components 180, 192, and 184 are regarded as part of manipulator system 200, since those base and slide components provide one of the means of “orthogonal control” (described below) over an electrode, blade, needle, or other instrument affixed to the manipulator arm 270. However, it should also be recognized that, since the upper portions of the manipulator system 200 can be easily detached from the slide component 180 by using release screw 204, some users regard only the portions of the system above the slide 180 as the manipulator system. This is an arbitrary semantic distinction; so long as the reader recognizes that the manipulator slide provides one of the three types of orthogonal control over an instrument, it does not matter whether that person considers the manipulator slide to be part of the manipulator, or not.

The upper portions of manipulator assembly 200 are detachably mounted on one end of manipulator slide 180, under the control of clamping screw 204. If clamping screw 204 is loosened, manipulator assembly 200 can be detached from the sliding base 180 and U-frame 104, for purposes such as cleaning, replacement by a different manipulator assembly, etc.

A rounded base component, referred to herein as turret base 202, allows the upper components of manipulator assembly 200-DIG to be rotated, in either direction, about a vertical axle, while sitting on top of manipulator slide 180. This allows the manipulator assembly 200 to be rotated until its upper portions (and an instrument, if one has been affixed to the V-block 290) are out of the way; this can be highly convenient during certain stages of a procedure, such as while an animal is being secured to or removed from the holder, and while an animal is being surgically prepared for a procedure.

To ensure proper alignment of the manipulator while in use, turret base 202 is typically provided with an etched mark that rotates with the base, positioned directly above an etched scale that is mounted on the end of manipulator slide 180. This scale (not shown in FIG. 1 or 2, since it is usually positioned on the outside of the sliding base 180) typically indicates degrees of rotation, and typically has an enlarged center mark, indicating “orthogonal” alignment of the manipulator arm when the etched mark in the base aligns with the center mark in the scale.

Additional positioning control of the manipulator assembly 200 can be provided by horizontal (medial-lateral) axle 205, which allows rotation when axle screw 206 is loosened. This allows the upper portions of the manipulator assembly to securely affixed at any desired vertical angle.

In some stereotaxic holders, a squared coupling base 207 is also provided. If clamping screw 208 is loosened, this will allow the upper components of the manipulator assembly 200 to be lifted slightly, rotated exactly 90 degrees, and affixed once again to the squared coupling base 207. This moves the manipulator arm and instrument out of the way, allowing various surgical, observational, or other steps in the procedure to be completed without hindrance by the manipulator arm or instrument; then, after those steps have been completed, clamping screw 208 can be loosened once again, and the upper components of the manipulator assembly 200 can be lifted slightly and rotated once again, back into their exact previous position. Therefore, this method of temporarily moving the manipulator arm and instrument out of the way, in the middle of a procedure, offers a major advantage compared to the turret base 202. If a procedure is being performed where distances measured in microns are important, any rotation of turret base 202 would render exact repositioning of the manipulator 200 impossible.

The manipulator components that are located above the squared coupling base 207 can be regarded as forming several subunits or assemblies, which are referred to herein as block 260, and horizontal arm 270. As shown in FIG. 2, vertical arm 240 allows precise control of the vertical positioning of an instrument tip, by rotating knob 241. When an animal is in a conventional position, with its feet resting on baseplate 102, movement in the vertical direction is referred to as dorsal (i.e., upward, from the animal's feet toward its backbone) or ventral (i.e., downward).

Horizontal arm 270 allows precise control of the medial and lateral positioning of an instrument tip, by rotating knob 271, but those two terms are not used according to standard medical practice. In standard medical terms, “medial” travel refers to motion that approaches the center plane of an animal, while “lateral” refers to motion away from the center of the animal. However, neurological procedures on small animals use a point at the center of the skull (called the bregma, described below) as the baseline starting point for all distance measurements; therefore, travel in either direction, away from the bregma, would be called lateral. To sidestep that problem, the manipulator 200 can be regarded as a baseline starting point. This shifts the reference plane to the left of the animal, since manipulators are conventionally mounted on the left side of a stereotaxic holder (presumably to make access to animals easier, for right-handed people). Using this convention, the term medial refers to travel or positioning which is toward an animal's left side (i.e., closer to a conventional manipulator), and lateral refers to travel or positioning which is toward an animal's right side (i.e., farther away from the manipulator).

Returning to FIG. 2, the vertical arm assembly 240 comprises several distinct components. Threaded shaft 242, which is coupled directly to vertical control knob 241, is positioned between Vernier rod 248 and stabilizer rod 249. Rods 248 and 249 are both securely coupled, at both ends, to vertical end caps 250 and 251.

As threaded shaft 242 is rotated under the control of knob 241, two smooth bushings (these also can be called sleeves) 256 and 258 slide along the smooth shafts of smooth rods 248 and 249. These bushings, typically made of a hard plastic such as nylon, DELRIN™, etc., are mounted inside a travelling support block 260, which supports the entire horizontal arm assembly 270. An internally-threaded bushing (sleeve) 244 is also affixed, in a nonrotating manner, inside travelling support block 260. Since bushing 244 cannot rotate, it is forced to travel in a vertical position, upwardly or downwardly, whenever threaded vertical rod 242 is rotated under the control of knob 241. In this manner, precision control over the vertical motion of the travelling block 260 (and therefore of the entire horizontal arm 270 as well) is provided by control knob 241.

In a similar manner, horizontal arm 270 includes a threaded shaft 272, which rotates under the control of knob 271. Threaded shaft 272 is flanked by Vernier rod 274 and stabilizer rod 276. An internally-threaded bushing (not shown, positioned inside the travelling block 260) causes the horizontal arm assembly 270 (and any instrument affixed to it) to travel in a medial or lateral direction when the knob 271 and the threaded shaft 272 are rotated. While the horizontal arm 270 travels, Vernier rod 274 slides through a bushing 278 with a smooth internal surface, and stabilizer rod 276 slides through another smooth bushing (not shown) which is mounted inside travelling block 260.

At one end of horizontal arm assembly 270, located adjacent to knob 271, the Vernier rod 274 and stabilizer rod 276 are securely affixed inside end cap 280. At the opposed end of horizontal arm assembly 270, end cap or “V-block” 290 is provided with a V-shaped notch, as shown in FIG. 2, with an internally-threaded screw hole in its center. The notch and the threaded screw hole in V-block 290 work together to allow any desired type of instrument (shown generically as instrument 300, in FIG. 1) to be temporarily yet securely affixed to the V-block 290.

A typical instrument 300 includes a securing clamp 310, a vertical shaft 320, and an instrument head 330 at the lower end of shaft 320. A typical securing clamp 310 includes: (i) a horizontal bar with a V-shaped surface that fits into and accommodates the notch in V-block 290; (ii) a knob which rotates a threaded shaft that screws into the screw hole in V-block 290; and (iii) a rounded vertical clamp that fits around vertical shaft 320, and which is provided with a wing nut or similar tightening screw that can be used to tighten or loosen the vertical clamp, so that the vertical shaft 320 can be adjusted to any desired height and then secured at that height. Once a procedure has commenced, the vertical shaft is not moved or adjusted by manipulating securing clamp 310; instead, the height of the vertical shaft 320 is adjusted only under the control of the manipulator's vertical knob 241.

Any type of instrument (such as a blade, needle, electrode, etc.) that is desired for use in a particular type of procedure can be mounted to the lower end of instrument shaft 320, using (if desired) a mounting structure referred to generically herein as an “instrument head” 330.

Anyone who has used a stereotaxic holder, and anyone who examines FIGS. 1 and 2, will recognize how the three adjustment knobs 182, 241, and 271 work together to provide complete three-dimensional control over the exact placement (positioning) and movement (travel) of the instrument tip, at any given moment during a test on an animal.

The manipulator slide 180, the vertical arm assembly 240, and the horizontal arm assembly 270 are all positioned in an “orthogonal” arrangement; this means that each slide or shaft is perpendicular to the other two. Using standard “Cartesian” coordinates (named after the French mathematician Rene DesCartes), they establish three “axes” of motion, which can be designated as the X, Y, and Z axes, as shown in the lower left corner of FIG. 1.

Using the conventional directional terms used in neurology, the manipulator slide 180 and its knob 182 control motion of the manipulator along the “A-P” (anterior-posterior) axis. The vertical threaded shaft 242 and knob 241 control motion of the manipulator along the “D-V” (dorsal-ventral) axis. The horizontal threaded shaft 272 and knob 271 control motion of the manipulator along the “M-L” (medial-lateral) axis. These axis designations are shown in FIG. 1.

Many labs (and some instrument makers) also refer to these axes as the X, Y and Z axes, using the system that most students encounter in mathematics classes, in high schools. When X, Y, and Z designations are used, the medial-lateral axis is deemed to be the X axis, the anterior-posterior axis is deemed to be the Y axis, and the dorsal-ventral) axis is deemed to be the Z axis. These X, Y, and Z designations are also shown in FIG. 1, and in various items of prior art, such as U.S. Pat. No. 6,258,103 (Saracione, 2001).

As noted above, the type of stereotaxic holder which is illustrated in FIG. 1 is well-known prior art; similarly, all components shown in FIG. 2 which have callout numbers between 100 and 399 are prior art. Thousands of stereotaxic holders having this arrangement (or very similar arrangements) have been sold; they are standard equipment in nearly any neurology lab that works with surgical or other invasive procedures on small animals. The only components which are new, and which help illustrate this invention in FIG. 2, have callout numbers higher than 500.

In neurological tests on small animals, it is often necessary to establish the exact location of an electrode or other instrument tip, in the brain. Since minor variations arise between different animals in skull thickness and other anatomical structures, positioning inside the brain is usually measured relative to a certain point, called the “bregma”, which is visible on the top surface of the skull of a rat or mouse. As can clearly be seen by looking at a rat skull, the top surface is formed when several bone structures, usually called “plates”, fuse together to form a larger single structure. The remnants of the different plates remain visible, and are separated by shallow zig-zagging crevices between the plates. These crevices are usually called “sutures”, since they resemble stitches made of thread, or “fissures”, a term which refers to a furrow or crevice between two adjacent objects.

Since two anterior plates (left and right) merge with two posterior plates (again, left and right), two major fissure lines (called the sagittal suture, in the anterior-posterior direction, and the coronal suture, in the medial-lateral direction) cross and intersect with each other, in a generally “+” configuration.

This point of intersection, where the two major fissure lines cross each other, is called the bregma. It has a physical appearance similar to the “cross-hairs” used in rifle scopes, and in many types of camera viewfinders, microscopes, and telescopes. Its vertical position is established when the tip of an electrode or other instrument is lowered down onto the skull until the instrument tip barely touches the intersection of the two fissures.

Another important physiological location on the skull is called the “lambda”. This is another easily visible skull suture, which is posterior or “caudal” (i.e., closer to the tail) from the bregma. In many types of tests, the animal's head must be oriented in a “flat skull” position, which indicates that the vertical height of the bregma and lambda locations must be the same. This can be accomplished by loosening the snout clamp slightly, adjusting it up or down, and tightening it again, while the ear bars remain firmly in place to establish an axis of rotation.

As mentioned above, the bregma is regarded as a “zero point” location, in neurological tests on small animals. All other locations are described by indicating their distance and direction from the bregma, along each of the three axes. Distances along all three axes must be indicated, to establish an exact location inside a brain. As an example, in the brain of a typical adult male rat weighing 250 grams, the center of the ventomedial nucleus of the hypothalamus would have coordinates of ML+0.5, DV−3.6, and AP−4.6 (all in millimeters). Three-dimensional maps or “atlases” of rat brains have been published, with enlarged photo-micrographs of the brains at various coordinates, and indicating the appearances of various structures within the brain. One such atlas can be downloaded over the Internet, from http://java.usc.edu/cgi-bin/HBPReg/webdriver?MIval=index.html&tool=3DBrainAtlas.

In conventional stereotaxic holders, the distance of any specific location, from the bregma point on the top surface of the skull, must be measured and calculated in a manner often referred to as “manual” or “analog”. This requires a cumbersome, awkward, and time-consuming procedure, which requires the operator to carefully and closely examine the stereotaxic holder from three different angles. That task can be very difficult, especially if a test is being carried out on a crowded laboratory bench or under a hood, and it is prone to introducing errors into the measurements.

Briefly, the manipulator slide 180 and both of the nonthreaded Vernier rods 248 and 274 (all of which are movable) are each provided with a linear scale, typically divided into centimeters and millimeters. This linear scale travels next to a non-moving reference mark, positioned directly alongside the moving scale. One such scale system is shown in FIG. 2, as scale 262, located in a “window” in travelling block 260. By reading the exact position of all three movable scales, in relation to each of the three reference marks, the complete three-dimensional location of the manipulator's V-block 290 (and any instrument tip which is securely affixed to V-block 290) can be measured, at any given moment. All three “coordinates” can be written down or typed into a computer or other electronic device, at any desired location or moment in time. Each recorded value can then be compared to the corresponding value on the same axis that was recorded earlier, when the instrument tip reached the bregma location. Then, by a process of subtraction, the distance of the instrument tip, at a location of interest, can be calculated in terms of how far it is from the bregma location that was recorded earlier.

This process is tedious and complicated, and it is rendered even more complex and difficult by the use of “Vernier” scales. These are difficult to describe in words, and they are also difficult to use, especially for people who do not use them frequently. Briefly, in a Vernier scale which uses 1-millimeter spacing, the “baseline” or zero mark on the non-moving component is accompanied by 10 additional marks, which have 0.9 mm spacing. By determining which of the 0.9-mm-spaced-lines is lined up most directly and exactly, across from a mark on the other aligned scale which uses exact 1 mm spacing, a skilled operator can determine (or at least closely approximate) the actual measurement, down to a fairly reliable 0.1 mm value.

For example, if the zero or “baseline” mark on the Vernier sale lines up between the 3 and 4 millimeter marks, and the sixth mark away from the zero/baseline mark on the 0.9-mm-spaced Vernier scale lines up exactly across from a mark on the 1.0-mm-spaced scale, then the actual value is very close to 3.6 millimeters.

Several major difficulties are encountered when stereotaxic holders are used in neurological research on rats or other small animals. The purpose of this invention is to provide a useful and highly convenient device and method for addressing and overcoming these difficulties, at minimal cost and by means of components which can be retrofitted onto most types of conventional stereotaxic holders in use today.

The first major difficulty relates to the inability of conventional stereotaxic holders to provide a simple method of establishing and recording the “zero point” that is determined when the instrument tip is positioned precisely at the bregma location. As briefly noted above, in most types of tests, the bregma location must be recorded and stored, so that all subsequent locations can be determined by referring to distances (along all three axes) from the bregma. In most laboratories, this recording step is typically done physically, by using a pencil or pen to write all three numbers (i.e., the X, Y, and Z axis values) on a piece of paper, such as a worksheet form, or a page in a laboratory notebook. Then, the three coordinates which will indicate the exact location(s) of the instrument tip at subsequent times or steps of interest, must also be measured and written down, so that the subtraction calculations can be performed to indicate the actual location of the instrument tip at those times or steps of interest.

Clearly, that series of steps which must be taken to determine the location of a brain structure tend to be tedious and disruptive, and they also subject the results to risks of errors of measurement, and errors of calculation. These problems are further aggravated by the use of Vernier scales, as briefly described above, since Vernier scales require close visual examination (which require very sharp eyesight at close range, since distances measured in tenths of millimeters must be measured; this can be rendered even more difficult by corrective lenses and/or safety glasses), and also require a careful mental calculation, before a single number can be written down based on where the scale stands. It should also be borne in mind that three different Vernier scales must be examined carefully and mentally calculated, three different readings must be written down, and three different subtraction calculations must be carried out, for each and every location of interest.

It should also be recognized that the problems of learning to use these types of complex devices (and then using them reliably and consistently, even when their use may be only infrequent and sporadic) are even more difficult, if the operator does not have a solid and convenient command of the English language. This is the case among large numbers of laboratory investigators and technicians; as anyone who has recently worked in or toured any biological research laboratory can attest, there will almost always be substantial numbers of workers present who do not speak English as their native language. Clearly, their ability to handle the chores of mentally translating between different languages is rendered more complicated and difficult when they must use tedious mechanical systems to visually measure and then manually record numerous data points, which in the final analysis indicate nothing more than positioning, and which must be overlaid on top of the cellular, chemical, or other scientific data that are being created or gathered as the primary focus and real goal of the experiment being carried out.

Clearly, it would be simpler, easier, and more reliable, and less tedious, time-consuming, confusing, and distracting, if a convenient and inexpensive yet reliable device and method could be provided, for establishing a “zero value” for each of the three axes, when the instrument tip reaches the bregma.

In addition, it would provide numerous advantages if the current analog, Vernier, manual system could be replaced by an easily-readable, large-digit, digital readout, which could clearly and unmistakably indicate the location of the instrument tip in all three of the X, Y, and Z axes, at any moment in time.

Just as importantly, it would be highly useful if a stereotaxic holder system were provided that could measure distances in microns, or fractions of microns, rather than in millimeters or tenths of millimeters. A single millimeter is equal to 1000 microns; therefore, even if a Vernier scale can be used to reliably measure things down to 1/10 of a millimeter, that is still 100 microns. Since typical mammalian cells have diameters of about 10 microns or less, and since neurons have numerous long thread-like extensions (including axons, dendrites, and synaptic processes) which have even smaller diameters, a 0.1 mm scale cannot distinguish between individual neurons, as is often required or highly advantageous in various procedures, such as procedures involving the use of so-called “patch clamps”, which are highly specialized and highly miniaturized electrodes which can measure each nerve impulse received or transmitted by a single neuron.

For all of these reasons, it would be highly useful to have a stereotaxic animal holder which could provide both: (i) digitized data on the location of the instrument tip, at any time, coupled with (ii) simple “zero-ing” capability, so that the location of the instrument would be displayed in absolute numbers, relative to a bregma location which was set to zero values in all three axes.

Indeed, such a device has already been created, with the aid of government funding. It is commercially available, from a company called Cartesian Research, Inc., located in Sandy, Oreg. (www.cartesianresearch.com).

However, that system suffers from three important drawbacks and limitations, which severely limits its use in actual research. First, it is relatively expensive; as of October 2001, the smallest stereotaxic holder having that type of digital measuring and zeroing capability sold for $11,600. Second, it is relatively large and bulky; its total “footprint” size (i.e., the amount of area it takes up, on a laboratory benchtop or desk surface) appeared to be between 3 and 4 square feet. And third, it cannot be easily retrofitted onto existing stereotaxic holders; the complete system must be purchased “from the ground up”, even if the purchasing lab already has one or more stereotaxic holders and merely wants to upgrade those to a digital measuring and recording system.

Accordingly, one object of this invention is to provide a convenient and inexpensive digital system for measuring instrument locations, for use with stereotaxic animal holders.

Another object of this invention is to provide an inexpensive digital system for measuring instrument locations, which can be retrofitted onto existing stereotaxic holders.

Another object of this invention is to provide an inexpensive digital system for measuring instrument locations, which can provide a simple and convenient “zeroing” functioning when the instrument tip is positioned at a targeted reference point such as the bregma.

Another object of this invention is to provide an inexpensive digital system for measuring instrument locations at a resolution of 20 microns or less, and preferably at a resolution of 5 microns or less.

These and other objects of the invention will become more apparent through the following summary, drawings, and description of the preferred embodiments.

### SUMMARY OF THE INVENTION

A convenient and inexpensive digital system is provided, for digitally displaying precise instrument locations while a procedure is being performed on a rat, mouse, or other small animal in a stereotaxic holder. This system, which can be retrofitted onto the manipulators of most existing stereotaxic holders, uses three linear scaling devices (such as optical diffraction gratings or laser interferometers, or electrical capacitance systems), mounted orthogonally (i.e., along each axis of movement of a manipulator). Three electronic reader heads are also used. Each reader head continuously emits an analog signal, indicating its position along the length of the adjacent linear scaling device. The three analog signals from the three reader heads are converted into digital signals, which are sent to a computer, or to a dedicated device such as a small display box with three readout panels. Three data points are displayed simultaneously, in easy-to-read form, by the digital display device, indicating the location of the manipulator tip along the X (medial-lateral) axis, the Y (anterior-posterior) axis, and the Z (dorsal-ventral) axis. An inexpensive system disclosed herein provides a resolution of 5 microns, which is roughly half the diameter of a typical cell; finer resolutions (such as down to 1 micron) can be provided, if more expensive components are used. Since the digital display unit is separate from the stereotaxic holder unit, it can be placed in any convenient benchtop or shelf location, or mounted on a wall (such as next to a safety hood or glovebox).

This system also provides a simple and convenient “zeroing” function, which can set any or all of the X, Y, and/or Z values to zero, whenever desired by an operator. This allows much faster, simpler, and more reliable measurements of instrument tip locations relative to a baseline reference point, such as the bregma of an animal skull.

“Data-grab” capabilities can also be provided, allowing the system to record and/or print the coordinates of the instrument tip at any step or time, whenever activated by an operator. If a computer is used, it can allow much more complex gathering, storage, and manipulation of the data, by using various control buttons or touch-free sensors to activate or terminate one or more programmed operations at any desired step or time during a procedure. Computerized control of a manipulator and instrument can also be provided if desired, by using a computer to control electric currents that will drive small motors coupled to the manipulator.

Since the scale and reader components mounted on the manipulator are not bulky or cumbersome, they will allow the use of an optical microscope during a procedure; they also will allow a video camera mounted above the stereotaxic holder to provide real-time images on a nearby video monitor, with any desired level of magnification.

### BRIEF DESCRIPTION OF THE DRAWINGS

FIG. 1 is a perspective view of a conventional stereotaxic holder as used in the prior art, showing the base, ear pins, and snout clamp for holding the animal. This drawing also shows a standard type of manipulator, bolted to the U-frame of the holder device. Motion of the manipulator system is under the control of three knobs, each of which can be used to rotate a threaded shaft in a manner that controls the location and travel of an instrument that has been mounted on the end of the manipulator's horizontal arm. Arrows in the lower left indicate the X (medial-lateral), Y (anterior-posterior), and Z (dorsal-ventral) axes.

FIG. 2 is a perspective view of a manipulator assembly, with components that allow a display box or computer to display the location coordinates of an instrument tip during a procedure. Electronic reader heads 514, 554, and 574 have been mounted on certain components of the manipulator, adjacent to linear scaling devices 502, 542, and 562. As the manipulator is operated, the reader heads will travel along the linear scales, and will emit electronic signals that indicate their position and travel relative to the linear scales.

FIG. 3 is a perspective view of a digital display box, showing a readout panel for each of the three axes, an input cable which will carry incoming data from all three reader heads on the manipulator, and an output port, which can be used to send data to a computer or other processor or instrument. Each display panel is accompanied by a zeroing button, to allow the location data to be reset easily to zero when an instrument tip reaches a reference location such as the bregma. Each display panel is also provided with a “select” or “send” button, which can initiate various types of data manipulation by a computer. The display box is also provided with a sensor-activator on its side, so that an operator can trigger a zeroing, data-grab, data processing, or other action, by means such as moving a hand directly in front of the sensor without touching anything that might increase the risk of contamination.

FIG. 4 is an exploded view showing the main components of an inexpensive system using a reader head and an etched optical scale, which can provide resolution down to 5 microns. Three scale-and-reader combinations will be used with a stereotaxic holder, to provide location data for the instrument tip in all three orthogonal axes.

FIG. 5 is a schematic diagram indicating a manipulator system with reader heads that are connected by a data cable to a 3-channel analog-to-digital (A/D) converter box 920. This converter transfers signals from the three reader heads to a computer, which will display the instrument location coordinates on its monitor, and which can also carry out further processing of the data, if suitable software is provided. If desired, converter device 920 can also provide an interface which will allow computer-controlled activation of small electric motors which have been coupled to the threaded shafts of a manipulator; this can allow computer-controlled motion and positioning of the manipulator and instrument.

### DETAILED DESCRIPTION

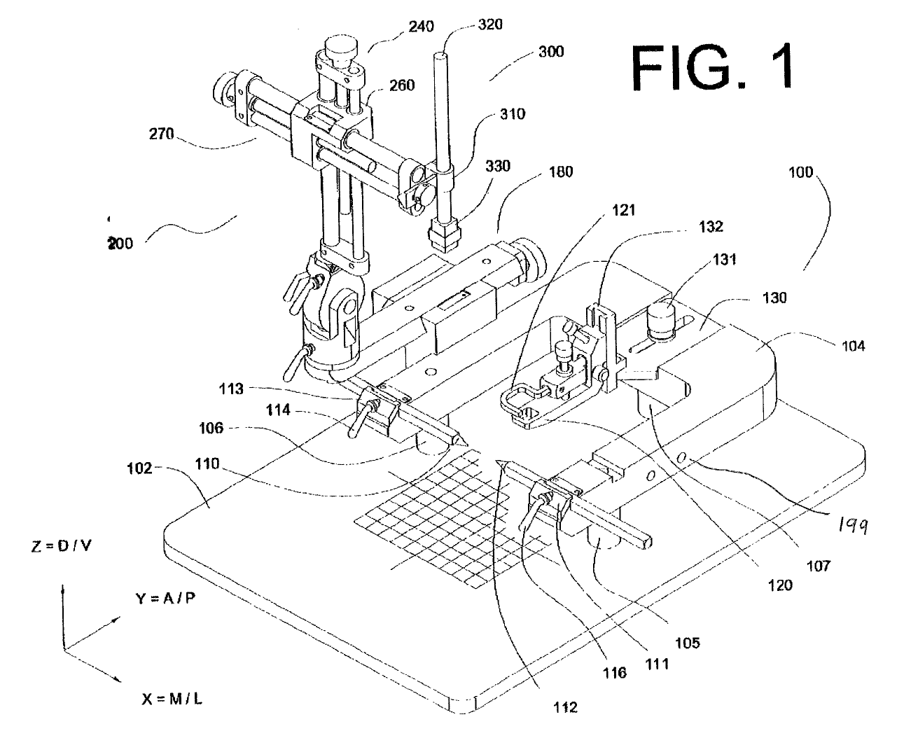

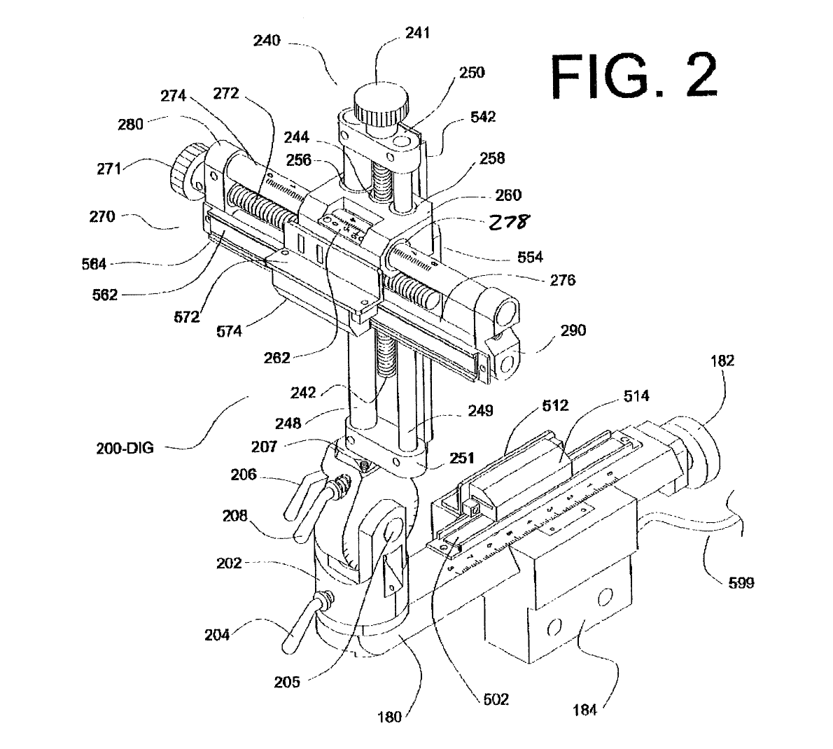

Referring to the drawings, a conventional stereotaxic holder unit 100, as shown in FIG. 1, can be retrofitted with a digital-readout manipulator system 200-DIG, as illustrated in FIG. 2, in either of two manners. In one mode, manipulator system 200 is removed, by unbolting it from the U-frame 104, and it is replaced by a different manipulator system 200-DIG, which has been fabricated as a complete unit. This unit can be securely affixed to the stereotaxic holder unit 100 by bolting or otherwise coupling the manipulator base 184 of the digital system 200-DIG to a conventional U-frame 104.

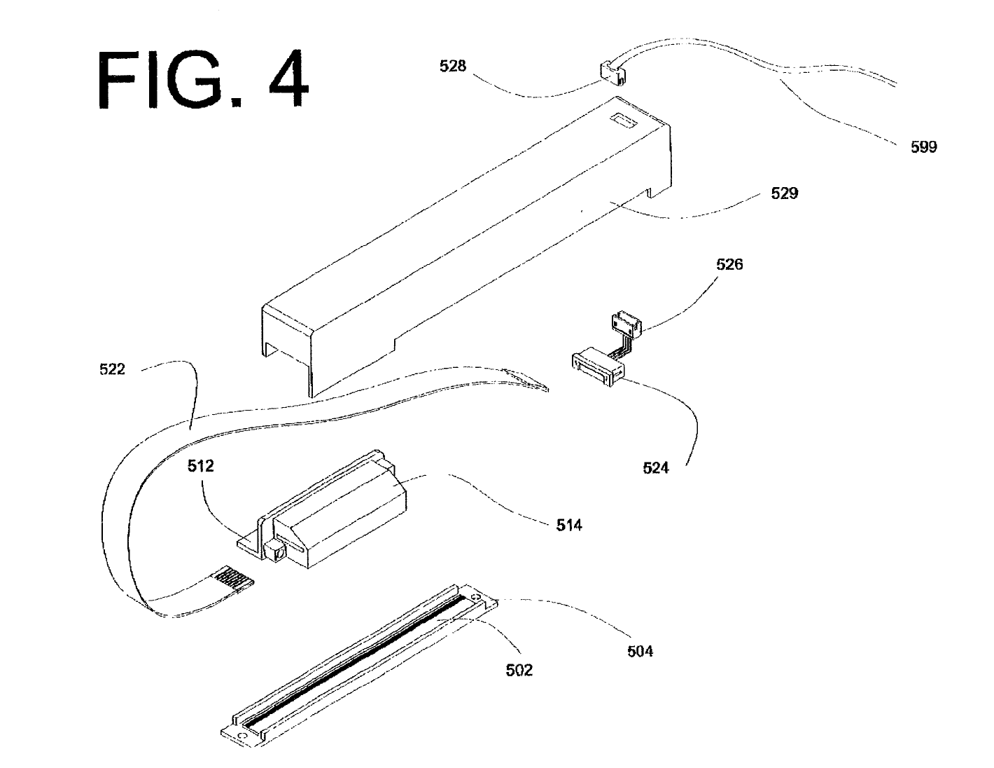

In another mode, a conventional manipulator 200 which has been removed from a conventional holder 100 can be retrofitted with scale-and-reader components, which are described below and which are given callout numbers 502 and higher in FIGS. 2 and 4. If proper kits and instructions are provided, and if an on-site machine shop has the right equipment, this might be done by a skilled machinist at a teaching hospital or other research institute. Alternately and preferably, it can be done by returning a conventional manipulator 200 to a shop which specializes in such retrofitting, by machinist-technicians who already know the parts and procedures involved, and who can check out the results and make any necessary adjustments using precision testing and calibration equipment.

Referring to FIGS. 2 and 4, the primary components of the digital measuring system which are mounted directly on the modified manipulator assembly 200-DIG include a linear scaling device 502 and a reader head 514, mounted on manipulator base 184 and slide 180 in a manner which causes them to move, relative to each other, as the slide 180 is operated. Reader head 514 provides location data along the anterior-posterior (Y) axis. A second linear scale 542 and reader head 554 pair are mounted on vertical arm 240, and provide location data along the dorsal-ventral (Z) axis. A third linear scale 562 and reader head 574 pair are mounted on horizontal arm 270, and provide location data for the medial-lateral (X) axis.

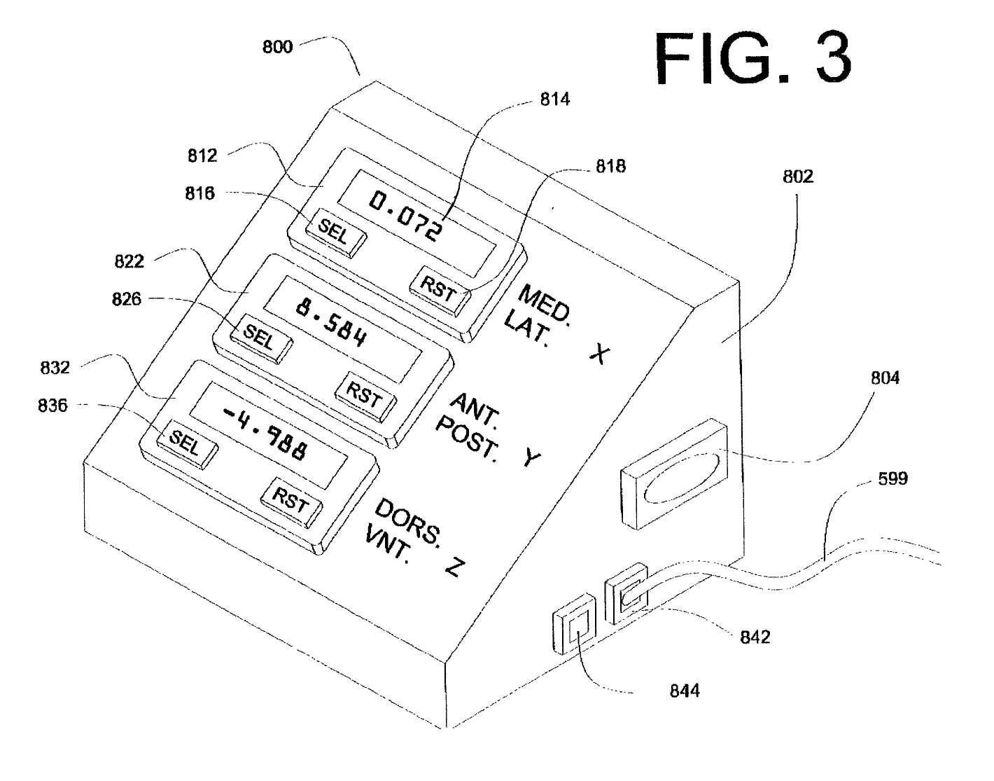

In one preferred embodiment, each of these three paired scale-and-reader units sends electronic data (normally in analog form, although it may be possible to use reader heads which emit digital signals) to a small and relatively inexpensive display box 800 (shown in FIG. 3), which simultaneously displays location data for all three orthogonal axes. This is done by using internal electronic components which generally include a 3-channel analog-to-digital (A-D) converter, at least one integrated circuit, embedded software, and memory capacity, all of which are discussed in more detail below. Data input port 842 (which is adapted to receive data from the manipulator 200-DIG) and an optional data output port 844 (which can be used to send digital signals to a computer, for recording or further processing) are shown on side wall 802 of box 800, solely for illustration; these data ports typically will be placed on the back of the box, so that the wires will be kept out of the way.

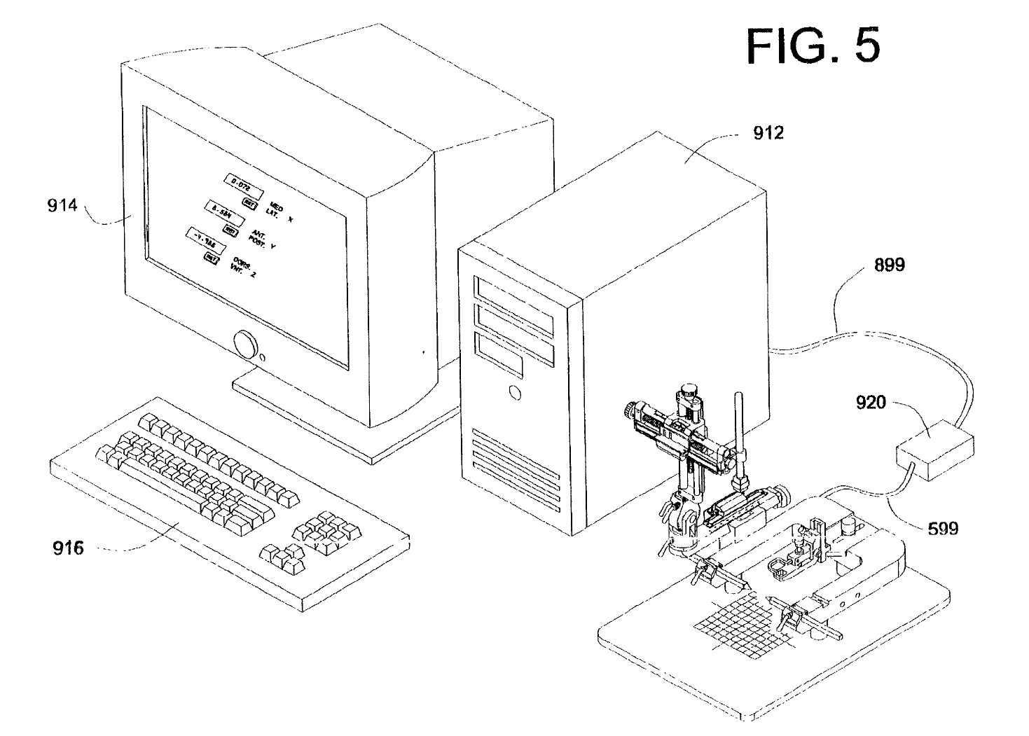

In an alternate preferred embodiment, illustrated in FIG. 5, the display box 800 can be eliminated, and a programmable computer 912 with a monitor 914 and keyboard 916 can be used to display orthogonal location data from the three scale-and-reader units on the manipulator system 200-DIG. As used herein, the term “computer” refers to a programmable machine that allows an operator to easily change software instructions, and that can display graphics on a monitor. Examples include conventional desktop or laptop computers, larger computers, and various programmable calculators or “personal digital assistants” that can display graphics. By contrast, terms such as “display box”, “processor”, or “dedicated device” describe devices in which (i) the software has been embedded into integrated circuits (which may include “EPROM” or similar programmable chips) and cannot be easily reprogrammed; and/or, (ii) the display outputs use simple and inexpensive panels to display numbers, rather than monitor screens that can display graphics. Display box 800, shown in FIG. 3, is an example of a dedicated device which is fairly small, rugged, and inexpensive.

If a computer monitor 914 is used as shown in FIG. 5, the analog signals from the three reader heads typically will need to pass, via data cables 599 and 899, through an analog-to-digital signal converter which can handle at least three “channels” (i.e., three separate and distinct signals, regardless of how many different wires, leads, or circuits are used to carry them). In FIG. 5, this type of signal conversion is provided by a stand-alone device 920 (which can be mounted on or near the manipulator base, if desired). Alternately, A/D conversion can be provided by an interface card which can be put into an expansion slot inside computer 912; such a card would be similar to a typical “sound card” which can convert two-channel analog stereo signals into digital signals that can be played or otherwise manipulated by a computer.

Since one of the goals of this system is to provide low-cost components that can be adapted to, or retrofitted onto, existing models of stereotaxic manipulators, it should be recognized that the components illustrated in FIGS. 1, 2, and 4 are well-suited for retrofitting onto conventional manipulators. To illustrate how this can be accomplished, mounting bracket 564, which holds etched scale 562 on the horizontal arm assembly 270, can be mounted at a suitable distance (or “clearance”) away from the surfaces of end blocks 280 and 290, by placing one or more flat washers or other spacer devices between the ends of the mounting bracket 564 and the end caps 280 and 290 of horizontal arm 270. The clearance gap, which can be set to any desired distance by simply using a desired number of washers and screws having sufficient length, should allow the horizontal arm assembly 270 to travel horizontally, without having the back side of mounting bracket 564 rubbing or scraping against the travelling block 260. The mounting bracket 572, which holds reader head 574, can also be given any desired dimensions (and can be provided with elongated slots instead of round screw holes, if desired, to allow further adjustments during installation) to cause the reader head 574 to remain at a desired spacing from etched scale 562 as etched scale 562 travels beneath reader head 574.

This same approach, using mounting brackets in combination with flat washers, can be used to attach the two ends of a vertical etched scale bracket to end caps 250 and 251 on vertical arm assembly 240, to allow unimpeded motion of the travelling block 260 along the vertical arm assembly 240.

If necessary, spacer-washers can also be used to provide clearance-controlled attachment of the scale 502 and reader head 514 to the manipulator base 184 and slide 180.

As used herein, the term “orthogonal” indicates that measurements and/or calculations are being made in three different axes (or directions, vectors, etc.), each axis being perpendicular to both of the other two axes. Essentially all stereotaxic holders used with animals are built with components that align very closely (within small margins of manufacturing tolerance) with the A-P, M-L, and D-V axes, as illustrated in FIG. 1.

However, if one wanted to create a “skewed” system, one or more of the “arms” could be placed at a slight angle away from true perpendicular (such as, for example, 85 instead of 90 degrees away from either or both of the other axes). In that type of situation, true and accurate orthogonal measurements can still be made, by mathematically applying sine or cosine values to the measured/apparent results, to obtain adjusted/corrected results.

Accordingly, “orthogonal” as used herein covers any system (including a deliberately angled or skewed system) that is designed to be capable of generating accurate measurements along the three axes conventionally used in neurological tests on animals (i.e., the anterior-posterior axis, the medial-lateral axis, and the dorsal-ventral axis).

On the subject of angled systems, it also should be noted that the system disclosed herein can be adapted to allow controlled and precise angling of a manipulator assembly, using either or both of two existing mechanical axles to allow partial rotation. A vertical mechanical axle passes through the center of turret base 202, discussed above; this axle is locked when clamping screw 204 is tightened. A horizontal axle, oriented in the medial-lateral direction, is provided by axle 205, which is controlled by clamping screw 206. If desired, yet another horizontal axle could be provided, in the anterior-posterior direction, by converting the single-axle joint on top of turret base 202 into a “gimbal” or “universal” joint.

If desired, various components that surround these mechanical axles can be provided with etched scales and reader heads, which will allow precise digital measurements of the rotation of a manipulator about any mechanical axle. As an example, an etched scale could be placed on the rounded end of manipulator slide 180, and an electronic reader head could be placed on the lower rim of rotatable turret base 202. The electronic signals from the movable reader head could be fed into a converter, such as exists in display box 800, and the resulting digital measurements could be fed into a computer, which could carry out any necessary trigonometric or other mathematical calculations, using automated software which would allow the final position of the instrument tip to be calculated and displayed, as a function of all of the reader head outputs (including any orthogonal reader heads, and any rotational reader heads), at any given instant.

Four factors should be noted about data cable 599, which transfers electronic signals from the three reader heads 514, 554, and 574 to display box 800. First, the multi-lead (or “trunk”) portion which connects with display box 800 will need to be split into three distinct branches, so that a set of leads (each lead comprising at least one insulated wire that can carry current) will physically travel to each of the three reader heads. This can be done by using “split cables” (which are commonly used in computers), by using a wiring harness for different cables, by using a reinforcing collar which is placed adjacent to a location where outer insulation is removed and smaller insulated leads branch out in different directions, or by any other suitable means.

Second, there is no particular fixed number of leads that must be connected to each reader head. If two leads are used for each reader head, they can provide a relatively low direct current voltage (such as 5 volts) to the reader head, while also carrying signals from the reader head, in the same manner that two-lead wires are used in conventional hard-wired telephones to provide power to a telephone set as well as carry the signals to and from the phone. Alternately, if three leads are used, one lead can be a “ground” lead, while one lead carries the power voltage and the other lead carries the signal. As another alternative (which is possible, but not preferred), the “ground” lead can be provided by the metallic components of the manipulator itself, and a single lead can be used to carry both the power voltage to, and the signal from, a reader head. As yet another alternative, four leads can be used, wherein two leads will carry power to the head and the other two leads will carry the signal from the head.

Third, there is no particular fixed number of leads that must enter the display box 800. As one example, if somewhat more complex electronic components are provided at or near the manipulator system 200-DIG, it would be possible to send electronic signals from all three reader heads through a single lead, using different “channels” or frequency ranges, in a manner analogous to sending numerous television channels through a single coaxial cable. This is not a preferred option, since it would increase the cost and complexity of the system, and the number of electronic components that might malfunction and/or be difficult to set up properly and/or diagnose. Keeping costs low, and keeping the system simple, easy to set up and use, and easy to diagnose if a malfunction occurs, are among the major goals and benefits of this system; accordingly, a simple and inexpensive multi-lead cable, having two or more leads for each reader head, offers a generally preferred approach.

The fourth factor is this: although a “hard-wired” data cable from the manipulator system 200-DIG to the display box 800 provides the simplest and least expensive means for data transfer, it is not the only data transfer means that can be used. If desired, wireless data transfer components can be used. One such system could use infrared (IR) beams, as used in typical remote controls for televisions and VCR's; however, these generally are not preferred, since they normally require an unblocked line-of-sight pathway for an infrared light beam from a transmitter to a receiver. A better candidate system could use radio-frequency (RF) signals, as used in typical cordless phones inside a house; these do not require an unblocked line-of-sight pathway between a transmitter and a receiver. IR and RF transmitter-and-receiver systems are common and relatively inexpensive, and they (or any other mode of wireless transmission currently known or hereafter discovered) can be adapted for use herein, to transmit electronic signals from manipulator system 200-DIG to display box 800. Nevertheless, they are not especially preferred, since: (i) they would add unnecessary expense to the system; (ii) they tend to make a system more difficult to diagnose and fix, if a malfunction occurs; and (iii) some form of power (voltage) must be provided to both the transmitter and the receiver. Even though voltage supply problems can addressed in various ways, such as by using a battery pack mounted on the manipulator, or a small transformer (often called a “wall wart”) plugged into a nearby 110-volt outlet, these types of “work-around” answers create further complications and suffer from various shortcomings. Accordingly, a simple multi-lead cable, between the manipulator and the display device, is generally a preferred mode for both: (i) supplying voltage to drive each of the three reader heads, and (ii) carrying data signals from each of the three reader heads to the display device.

If a stand-alone (“dedicated”) display box 800 is used, it typically will contain, within the box: (i) a three-channel A-D converter, or three distinct A-D converters, to convert the three analog signals from the reader heads into three distinct sets of digitized signals that can be processed by an integrated circuit; and, (ii) at least one integrated circuit, which contains (or is accompanied by) memory capacity that has been programmed with software. These electronic components interact with the software instructions, to convert the digitized signals from the A-D converter(s) into digital numbers that are displayed on the readout panels 812, 822, and 832, shown on the “face” side of display box 800. The readout panels can use any type of suitable numerical display components, such as liquid crystal displays 814, light-emitting diodes, etc.

The components of a scale-and-reader system (such as etched scale 502 and reader head 514, mounted on manipulator slide 180) are depicted in more detail in an exploded view, in FIG. 4. A protective cover 529, made of sheet metal, molded plastic, or similar material, is used to protect the etched scale and reader head from dust, droplets, scratches, and other hazards during use; for purposes of illustration, these covers are not shown over the scales or reader heads depicted in FIG. 2.

The components shown in FIG. 4 include: (i) etched scale 502, which is mounted on a mounting plate 504 to enable convenient handling and installation; (ii) reader head 514, which is mounted on a mounting bracket 512; and, (iii) a multi-lead data cable 522, which is inserted into a zero-insertion-force (ZIF) connector 524 that is coupled to connector socket 526. Connector socket 526 is designed to accommodate a connector plug 528, mounted at the end of data cable 599, which carries the electronic signals to display box 800.

All components used in these scale-and-reader systems can be purchased from commercial suppliers, such as Metrigraphics, a division of Dynamics Research Corporation (Wilmington, Mass.; www.drc.com). The electronic signals emitted by these reader heads can be read and interpreted by electronic equipment that is also available commercially, from companies such as Red Lion Controls (York, Pa.; www.redlion-controls.com).

The optical/electronic systems described below sound complex, when described in words; however, these components and the principles they use are well-known to those skilled in the art. These devices are mass-produced, and are sold commercially at very reasonable costs. Using simple mounting brackets, these subassemblies can be properly attached to a manipulator system, by drilling and threading accommodating screw holes at properly-targeted locations, on selected components of a stereotaxic manipulator.

In addition, it is not necessary that the scales or reader heads must be mounted and aligned with high precision, on a manipulator; instead, conventional aligning methods used by skilled machinists will be adequate for properly affixing the scales and reader heads to a manipulator. This is because a “zeroing” function is used to establish the starting or baseline position for each procedure, individually. Normally, this is done when an instrument tip touches the bregma, on an animal's skull. All subsequent measurements are relative, based on that starting point for that one particular animal, rather than on some “absolute” point that must be fixed and precise for all tests done with that holder unit.

When the display box is plugged in and turned on, a small voltage differential (such as 5 volts, direct current) between two of the wires within cables 599 and 522 will power the reader head 514. This voltage can be supplied by an electronic component inside display box 800; as mentioned above, this voltage can also be carried by the same two wires that will carry the electronic signal that emerges from the reader head 194.

When powered by this voltage, reader head 514 emits a small beam of light. The beam of light emitted by reader head 514 will reflect off of adjacent etched scale 502, and that reflection will immediately return to a sensing mechanism inside the reader head 514.

The term “light” is used herein for convenience, to refer to electromagnetic radiation. Most common lasers, light-emitting diodes, and similar devices that are convenient and inexpensive work within the visible spectrum. However, it should be understood that, if desired, a reader head can use an electromagnetic frequency outside the visible spectrum, such as in the ultra-violet range (in most optical systems, shorter wavelengths can generate more precise measurements).

Each reader head is positioned adjacent to a single etched scale; using the callout numbers shown in FIG. 2, reader head 514 is adjacent to etched scale 502, reader head 554 is adjacent to scale 542, and reader head 574 is adjacent to scale 562. Each scale-and-reader unit (or “couple” or “pair”) interacts and works together, as a single functional subsystem that allows the reader head to generate a usable output signal, during a test on an animal.

The scale and reader components of each unit must be mounted on two different components of the manipulator system 200-DIG, in such a way that either the etched scale or the reader head (but not both) will move, whenever the manipulator is operated by rotating one of the control knobs 182, 241, or 271 to change the position of the instrument tip along one of the orthogonal axes. This generates relative motion between the two scale-and-reader components which measure motion along that particular axis. As used herein, “motion” or “operation” of the manipulator, instrument, or instrument tip (and similar phrases, such as “during a test” or “during use of the manipulator”) refer to motion or travel of the instrument and instrument tip along one or more of the orthogonal axes.

The mounting arrangement shown in FIG. 2 is believed to offer a convenient and protective arrangement, since each elongated etched scale is mounted securely on an elongated component of a slide or arm. However, it should be noted that these mounting arrangements end up working in different ways. On manipulator slide 180 and horizontal arm assembly 270, reader heads 514 and 574 remain stationary while etched scales 502 and 562 travel; by contrast, on vertical arm assembly 240, the reader head 554 travels while the etched scale 542 remains stationary. Either type of motion is referred to herein as “relative” motion, where a scale will move relative to its adjacent reader head (or vice-versa), when a corresponding control knob 182, 241, or 271 is rotated.

During the operation of etched scale 502 and reader head 514, the beam of light which was emitted by reader head 514 and which has reflected off of etched scale 502 will be detected and processed by other electronic components inside reader head 514. By using a known type of device (such as or similar to a “diffraction grating” or “laser interferometer”), etched scale 502 is manufactured in a manner that provides it with a gradually changing “continuum of reflectivity”, which causes its reflective property to vary along the length of the scale, in a known, controlled, and precise manner. As a result, the intensity and/or wavelength of the reflected light which returns to reader head 514, at any particular position on the scale, will depend upon (and therefore indicate) the location of reader head 514 along the length of etched scale 502.

Changes in the intensity or wavelength of the reflected light which returns to reader head 514 will modify the intensity, wavelength, or other property of an analog output signal that is generated by reader head 514 and sent, via cable 599, to an analog-to-digital (A/D) converter, which normally will be located in a display box 800, a stand-alone converter unit 920, or a computer 912. Alternately, it may be possible to use electronic reader heads which directly provide digital signal outputs.

The electronic system inside display box 800 (which uses one or more integrated circuits and embedded software, all of which are well-known to the companies that manufacture such devices) allows the signal that emerges from reader head 514, to be converted into a digital number by the electronic components inside display box 800. That digital number, which will vary as the travelling etched scale 502 moves beneath the stationary reader head 514, will be displayed on the anterior-posterior readout panel 822, on the front of display box 800.

In other words, the travel of the scale-and-reader components, relative to each other, will generate a usable electronic signal that can be converted into a digital display number, expressed in millimeters (with three significant digits shown to the right of the decimal point, to indicate microns) or similar units. This same principle applies to scale 542 and its adjacent reader head 554 (relative motion between them can be converted into distance measurements on the D-V readout panel 832), and to scale 562 and its adjacent reader head 574 (relative motion between them can be converted into distance measurements on the M-L readout panel 812).

Since each reader head is mounted and wired independently of the other two reader heads, data from all three reader heads can be displayed, simultaneously, by display box 800. By combining the data generated by reader heads that are aligned with all three orthogonal axes, the exact coordinates of an instrument tip along all three axes, at any given moment during a test procedure, can be displayed digitally, in a clear, convenient, and easily-readable manner.

It should be recognized that etched scales and reader heads, as described above and as sold by companies such as Dynamics Research Corporation, are not the only type of electronic measuring systems that can convert distance measurements into digital readouts. As another example of a candidate system that might be adapted to use as disclosed herein, certain types of electrical capacitance systems are used on various types of measuring devices, such as calipers that have digital readouts. Digital calipers are sold by a number of companies, such as Competitive Edge Dynamics (www.cedhk.com), Chicago Brand (www.chicagobrand.com), and Mitutoyo (www.mitutoyo.com). In addition, various models of digital calipers have been developed with data output cables that are adapted to be plugged into computer interfaces, allowing computers to “grab”, store, and manipulate the measurement data in various ways.

If desired, such capacitance measuring systems (or any other type of known or hereafter-discovered electronic measuring system that can provide reliable linear measurements, to a resolution of about 5 microns or less) can be used as disclosed herein, for any or all of the scale-and-reader systems mounted on a stereotaxic manipulator system. The preferred type of measuring system (such as optical, capacitance, etc.) will depend on economic rather than technical factors.

If desired, printing capability can be provided directly within display box 800, by using a conventional miniaturized printer, as used by small adding machines or certain types of calculators. To avoid the need for inked ribbons or other complications, these can use small rolls of thermal- or pressure-sensitive paper. Printing of the location coordinates (along with the exact time of each such location printout, if a clocking circuit is also provided in the electronic components in display box 800) can be triggered whenever a “select” button is pressed.

Alternately, if desired, a zeroing, printing, or similar function can be triggered by a sensor device mounted on display box 800 at a convenient location, such as sensor device 804 mounted on one side of box 800. In one preferred embodiment, sensor device 804 can use means such as infrared rays to detect nearby motion of an operator's hand, without requiring anything on display box 800 to be touched by the operator. This type of “touch-free” activation can reduce the risk of contamination and infection of an animal being treated, and can also reduce the risk that blood, lymph, or cells that may be carrying pathogenic microbes might be smeared on the surface of display box 800. If desired, two or more touch-free sensors can be provided on a single box 800, by placing them on different sides of the box.

To eliminate the risk of inadvertently activating (“tripping”) a zero function when a button or sensor is activated, an activating routine can be programmed into the software. This might require, for example, two activating events within a brief span of time, in a manner similar to “double-clicking” a computer mouse.

Alternately and preferably, display box 800 can be provided with a data output port 844, which will allow the electronic components in box 800 to send the location data, via a data cable (or wireless means, such as an infrared or radiofrequency transmitter) to a nearby computer or other instrument (which can provide printing capability, thereby eliminating the need to provide printing capability as part of display box 800). This can enable much more sophisticated handling of location data that are sent to display box 800, as discussed in more detail below.

If desired, a backup battery or certain types of specialized memory chips can be provided in display box, to protect against loss of data in the event that the power supply to the display box is lost or interrupted.

Zeroing Function

As mentioned above, a simple and convenient “zeroing” function for each axis provides major advantages for this system, compared to the “manual” or “analog” systems that are in widespread use in prior art systems. This type of function can be provided, using integrated circuits and software that have been developed and incorporated into electronic systems designed for “display box” systems sold by companies such as Red Lion Controls. In this type of system, each digital display panel 812, 822, and 832, as shown in FIG. 3, can be provided with a “reset” or similarly-labelled button, positioned near the display panel. When this button is pressed (which most commonly will occur when an instrument tip reaches the bregma of an animal), the number shown on that display panel at that moment will be reset to a zero value (also referred to by terms such as a baseline, starting point, etc.). Until that reset button is pressed again, all subsequent numbers shown on that panel will be expressed as a distance relative to the location of the zero point. This will allow faster, simpler, and substantially more reliable measurements of the location of an instrument tip, relative to a fixed reference point such as the bregma.

If desired, the zeroing function for all three axes can be activated simultaneously, by touch-free sensor device 804.

To protect against inadvertent zeroing, which would seriously jeopardize the gathering of data during a test, any of several routines can be programmed into the software embedded in the integrated circuits inside box 800. As examples, a zeroing function might not be carried out unless a zeroing button is pressed three distinct times within two seconds, or unless all three zeroing buttons are pressed within two or three seconds of each other. Alternately or additionally, once a zeroing function has been triggered once during a procedure, the software can be programmed to prevent it from happening again, unless a “reset” routine is carried out which will not happen accidentally.

As shown by the negative value on display panel 832, all three display panels should be able to display negative values. Negative values will occur frequently, whenever a bregma or other reference point is used to determine the starting or baseline point for each axis.

Other Data Handling Functions

As mentioned above, display box 800 can be provided with a data output port 844, which will allow the electronic components in the box to send the location data to a computer or other instrument. Alternately, as illustrated in FIG. 5, the analog signals from the reader heads can pass through an A/D converter, and be sent directly to a computer, without requiring a dedicated display box. Either method can allow a computer to carry out far more complex and sophisticated gathering, storage, and manipulation of the location data than can be performed by a simple and inexpensive dedicated display box 800.

If this type of system is used, one or more buttons labeled as “Select”, “Send”, “Function”, “Activate”, etc. (or abbreviations thereof) can be placed at one or more convenient locations on the surface of the display box 800. Alternately, if an A/D converter with no display box is used as shown in FIG. 5, various keys on the computer keyboard 916 can be programmed to activate any of the zeroing or other steps listed below.

In the dedicated display box 800 illustrated in FIG. 3, three “Select” buttons 816, 826, and 836 are shown as components of display panels 812, 822, and 832, because such two-button display panels are already commercially available. If display box 800 is coupled to a computer via a data cable plugged into output port 844, each of these three “Select” buttons can be used to allow the operator to activate or terminate some predetermined operation, at any desired moment or step during a test, using various “switches” that have been written into the software that is running on the computer.

As an illustrative example, software can be written to allow any or all of four different data-handling functions to be triggered by buttons and sensors on display box 800 (and/or keys on computer keyboard 916). Such functions which are likely to be useful in a variety of tests can include, for example, any or all of the following functions:

(1) the computer will store and/or print out the location coordinates, in all three axes, at the moment when a button is pressed or a sensor is activated, along with a record of the exact second the activating signal was received (either in absolute time, or relative to a starting or baseline moment when the location coordinates were set to zero);

(2) the computer will begin gathering and storing continuous data on the travel, location, and velocity of the instrument tip;

(3) the computer will begin indicating, on the monitor screen and/or via one or more audible signals such as tones or digitized voices emitted by a speaker, the location, travel and/or velocity of the tip in any one or more of the X, Y, and Z axes; and/or,

(4) the computer will begin generating audible voices or tones emitted by a speaker, or numbers or other visual signals shown on the computer display monitor, that will help an operator guide the instrument tip to an exact desired location, or series of locations.

Alternately or additionally, Select, Send, or similar buttons can be used to commence a set of automated manipulations that will be guided by a computer. If desired, such manipulations can be programmed so that they can involve either or both of the following: (i) rotation of any or all of manipulator knobs 182, 241, or 271 by a small computer-controlled electric motor, thereby allowing automated and/or computer-controlled repositioning of the instrument tip; and/or, (ii) one or more activities carried out by an instrument tip, such as a blade, needle, or electrode which is contacting a targeted cluster of cells in an animal's brain or spinal cord.

Except for the above-mentioned option of computer-controlled activities by the instrument tip, the above-listed options all depend entirely on the location and/or travel of an instrument tip, as distinct from any chemical, electrochemical, or other measurements that can be made by the instrument itself. Any of these location functions or computer-controlled motion of the manipulator (and various others functions as well) would be useful in various types of tests that are carried out on animals being held by stereotaxic holders. Such data-gathering, data-manipulating, and manipulator-control functions, using a programmable computer or other electronic instrument, can be much more convenient, useful, and effective, if they can be activated or terminated merely by pressing a button, or waving a hand in front of a touch-free sensor.

In addition, it should be noted that since this digital measuring and readout system is not bulky or cumbersome, it allows the use of a stereo or other optical microscope, and/or the use of a video camera lens that can provide real-time images on a nearby video monitor, with any desired level of optical magnification. A separate yet related patent application will specifically address a more elaborate system which includes (i) a small video camera having a fairly powerful magnifying lens, mounted above the rear edge of the base plate 102, to allow the video camera to provide a clear perspective view of the top of the animal's head (including its skull and brain, if exposed); and, (ii) a moderately sized video display monitor, preferably having a diagonal dimension of about 20 cm to 40 cm (about 8 to 15 inches), to allow students, teachers, or other observers to clearly witness the progress of a test, which can also be recorded if desired for subsequent display or analysis. If this type of system is used, the exact location coordinates of the instrument tip, at all times, can be displayed in one or more corners of the video screen.

Finally, it should be noted that some types of invasive stereotaxic procedures use two (or even three or four) manipulator systems, mounted on opposite sides of the U-frame of a stereotaxic holder. A second manipulator can be mounted on a conventional U-frame, without requiring any substantial equipment changes, by bolting a second manipulator base 184 to right-side mounting holes 199, shown on U-frame 104 in FIG. 1. The entire vertical arm assembly 240 (along with the travelling block 260 and the horizontal arm 270) can be rotated, by loosening clamp screw 208, lifting out the square base 207 from its nest, rotating the assembly 180°, returning square base 207 to its nest, and tightening clamp screw 208. This rotation step will place V-block 290 (and any instrument affixed to it) directly over the work area, and work can proceed in the same manner as described above.

If dual-manipulator tests are desired, they can be done fairly easily, using the digital display system disclosed herein, merely by using a second display box. If dedicated display boxes are purchased and used by a lab, a display box normally will accompany any manipulator system that has been fitted with electronic reader heads.

Alternately, if a lab normally uses a programmable computer for its display, it can use either of two approaches to support dual-manipulator tests. In one approach, it can use a three-channel A/D converter and a computer (as described above) to handle data from one of the manipulators, while using a small and inexpensive display box to handle data from the other manipulator. Alternately, it can purchase an A/D converter having six or more channels, and use six channels to handle and display the location data from both manipulators. Multi-channel A/D converters have been developed (mainly for multi-track sound recording) and are commercially available.

Thus, there has been shown and described a new and useful device and method for providing improved controls, measurements, and displays, during tests on small animals in stereotaxic holders. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. An electromechanical system for digitally measuring and displaying location data for an instrument tip during a procedure on an animal being held by a stereotaxic holder, comprising:
(a) a manipulator system, having a base which can be securely affixed to the stereotaxic holder, and having an instrument attachment component to which at least one instrument having an instrument tip useful in procedures on animals can be securely affixed, wherein the instrument attachment component and an instrument affixed thereto can be moved in a controlled manner along each of three orthogonal axes, and having at least three electronic reader heads which are affixed to selected components of the manipulator system, wherein each reader head is positioned near an adjacent linear scaling device in a manner which will cause relative motion of the reader head with respect to its adjacent linear scaling device during motion of the instrument tip along a corresponding orthogonal axis, and wherein each reader head is designed to transmit, during use, electronic signals to a digital display device, said electronic signals being correlated to changeable locations of the instrument tip during a procedure; and,
(b) a digital display device, having (i) means for receiving electronic signals from at least three reader heads mounted on the manipulator system; (ii) data processing components and software, which together are capable of converting electronic signals originating from the reader heads into three independent and changeable orthogonal values expressed in digital form, said changeable orthogonal values being correlated to changeable locations of the instrument tip along orthogonal axes during an invasive procedure; (iii) means for displaying each of said orthogonal values, in either positive or negative form, in a manner clearly visible to a human operator,
wherein the digital display device is not rigidly affixed to the stereotaxic holder, and can be placed during use in a procedure in a location that is independent and separate from the stereotaxic holder.

2. The electromechanical system of claim 1, wherein at least one reader head is provided with first means to emit electromagnetic radiation toward an adjacent reflective linear scaling device having at least one reflectivity trait that varies in a controlled manner along its length, and second means for measuring electromagnetic radiation which reflects off the adjacent reflective linear scaling device.

3. The electromechanical system of claim 1, wherein at least one reader head uses a capacitance measuring system, to generate electronic signals which correlate to the reader head's position relative to an adjacent linear scaling device.

4. The electromechanical system of claim 1, wherein each reader head, when acting in conjunction with an adjacent linear scaling device and with the digital display device, is capable of displaying linear positioning along an orthogonal axis with a resolution of about 20 microns or less.

5. The electromechanical system of claim 1, wherein each reader head, when acting in conjunction with an adjacent linear scale and with the digital display device, is capable of displaying linear positioning along an orthogonal axis with a resolution of about 5 microns or less.

6. The electromechanical system of claim 1, wherein the reader heads and linear scaling devices are made of mass-manufactured components that can be retrofitted onto an existing manipulator system of a conventional stereotaxic holder.

7. The electromechanical system of claim 1, also comprising a flexible multi-lead cable which is capable of (i) supplying voltage to each of three reader heads in different locations; and (ii) carrying electronic signals from each of three reader heads to an electronic signal-processing device.

8. The electromechanical system of claim 1, wherein the digital display device has at least three display panels, each of which can display positive and negative digital values independently of the other two panels.

9. The electromechanical system of claim 1, wherein the digital display device comprises a programmable computer with a monitor.

10. The electromechanical system of claim 1, wherein the digital display device is provided with means to: (a) allow a human operator to set a zero value for at least one displayed orthogonal value, by activating at least one triggering mechanism when the instrument tip is at a predetermined baseline location; and, (b) subsequently display at least one orthogonal location value by indicating a measured linear distance of the instrument tip from the predetermined baseline location, at each moment during the procedure.

11. The electromechanical system of claim 1, wherein all components of the manipulator system are of a size, and are positioned in a manner, which allow a human operator to continuously observe an animal's head through a stereoscopic microscope, during a procedure on the animal.

12. The electromechanical system of claim 1, wherein all components of the manipulator system are of a size, and are positioned in a manner, which allow a video camera positioned above the center of the base plate's anterior edge to obtain and provide, to a display monitor, continuous video images of a procedure being carried out on an animal's head.

13. A stereotaxic manipulator system for providing digital displays of orthogonal locations of an instrument tip during a procedure on an animal being held by a stereotaxic holder, comprising:
(a) a manipulator base which can be affixed to a stereotaxic holder;
(b) an instrument attachment component which can be moved in a controlled manner along each of three orthogonal axes, by motion of interacting manipulator components;
(iii) at least three linear measuring systems, each comprising an electronic reader head mounted adjacent to a linear scaling device, in a manner which causes relative motion of the reader head with respect to the scaling device during operation of the manipulator system,
wherein the three linear measuring systems are oriented in a manner which effectively provides linear measurements along each of three orthogonal axes, and,
wherein each reader head is designed to generate electronic signals which (i) are correlated, at each moment during a procedure on an animal, with its position along its adjacent linear scaling device, to a resolution of about 20 microns or less, and (ii) can be converted to digital signals that can be processed and displayed by a digital display device.

14. The stereotaxic manipulator system of claim 13, wherein at least one reader head is provided with first means to emit electromagnetic radiation toward a linear scaling device having at least one reflectivity trait that varies in a controlled manner along its length, and second means for measuring electromagnetic radiation which reflects off the linear scaling device.

15. The stereotaxic manipulator system of claim 13, wherein at least one reader head uses a capacitance measuring system, to generate electronic signals which correlate to the reader head's position relative to an adjacent linear scaling device.

16. The stereotaxic manipulator system of claim 13, wherein each reader head, when acting in conjunction with an adjacent linear scale and with the digital display device, is capable of displaying linear positioning along an orthogonal axis with a resolution of about 5 microns or less.

17. The stereotaxic manipulator system of claim 13, wherein the reader heads and linear scaling devices are made of mass-manufactured components that can be retrofitted onto an existing manipulator system of a conventional stereotaxic holder.

18. The stereotaxic manipulator system of claim 13, also comprising a flexible multi-lead cable which is capable of (i) supplying voltage to each of three reader heads in different locations; and (ii) carrying electronic signals from each of three reader heads to an electronic signal-processing device.

19. The stereotaxic manipulator system of claim 13, also comprising a digital display device that has at least three display panels, each of which can display positive and negative digital values independently of the other two panels.

20. The stereotaxic manipulator system of claim 13, also comprising an analog-digital converter device which is capable of simultaneously converting at least three separate analog signals from the three reader heads into digital data which can be manipulated by a computer.

21. The stereotaxic manipulator system of claim 13, wherein all components of the manipulator system are of a size, and are positioned in a manner, which allow a human operator to continuously observe an animal's head through a stereoscopic microscope, during a procedure on the animal.

22. The stereotaxic manipulator system of claim 13, wherein all components of the manipulator system are of a size, and are positioned in a manner, which allow a video camera positioned above the center of the base plate's anterior edge to obtain and provide, to a display monitor, continuous video images of a procedure being carried out on an animal's head.

23. A method for providing a stereotaxic manipulator with electronic components capable of digitally displaying location data for an instrument during a procedure on an animal, comprising the following steps:
a. securely affixing, to at least two components of a sliding base of the manipulator, a first scaling device and a first reader head, in a manner which causes relative motion of the first scaling device with respect to the first reader head during operation of the manipulator slide;
b. securely affixing, to at least two components of a vertical arm of the manipulator, a second scaling device and a second reader head, in a manner which causes relative motion of the second scaling device with respect to the second reader head during operation of the vertical arm; and,
c. securely affixing, to at least two components of a horizontal arm of the manipulator, a third scaling device and a third reader head, in a manner which causes relative motion of the third scaling device with respect to the third reader head during operation of the horizontal arm,
wherein each reader head is capable of emitting electronic signals that can be processed electronically to create digital displays of location data that vary during a procedure on an animal.

24. The method of claim 23, wherein each reader head is provided with first means to emit electromagnetic radiation toward an adjacent reflective linear scaling device having at least one reflectivity trait that varies in a controlled manner along its length, and second means for measuring electromagnetic radiation which reflects off the adjacent reflective linear scaling device.

25. The method of claim 23, wherein each reader head uses a capacitance measuring system to generate electronic signals which correlate to the reader head's position relative to an adjacent linear scaling device.

26. The method of claim 23, wherein each reader head, when acting in conjunction with an adjacent linear scaling device, is capable of displaying linear positioning along an orthogonal axis with a resolution of about 5 microns or less.
